(19) United States
(12) Patent Application Publication
(10) Pub. No.: US 2019/0043501 A1
(43) Pub. Date: Feb. 7, 2019

(54) PATIENT-CENTERED ASSISTIVE SYSTEM FOR MULTI-THERAPY ADHERENCE INTERVENTION AND CARE MANAGEMENT

(71) Applicant: Elements of Genius, Inc., Mt. Pleasant, SC (US)

(72) Inventor: Jonathan E. Ramaci, Mt. Pleasant, SC (US)

(21) Appl. No.: 16/053,603

(22) Filed: Aug. 2, 2018

Related U.S. Application Data
(60) Provisional application No. 62/558,936, filed on Sep. 15, 2017.
(60) Provisional application No. 62/551,007, filed on Aug. 28, 2017.
(60) Provisional application No. 62/547,519, filed on Aug. 18, 2017.
(60) Provisional application No. 62/544,369, filed on Aug. 11, 2017.
(60) Provisional application No. 62/540,456, filed on Aug. 2, 2017.

(51) Int. Cl. G10L 15/22; G16H 40/63; G16H 50/20; G10L 15/30; G10L 15/18; A61B 5/00

(52) U.S. Cl. CPC G10L 15/22; G16H 40/63; G16H 50/20; G10L 2015/223; G10L 15/1815; A61B 5/4803; A61B 5/486; G10L 15/30

(57) ABSTRACT

A patient-centered assistive system for multi-therapy adherence intervention and care management. The intervention may be implemented in the form of a wearable device providing one or more features of medication adherence, voice, data, SMS reminders, alerts, location via SMS, and 911 emergency. Embodiments of the present disclosure may function in combination with an application software accessible to multiple clients (users) executable on a remote server to provide patient education, support, social contact, management of daily activities, safety monitoring, symptoms management, adverse events reporting, as well as support for caregivers, and feedback for healthcare providers during the management of patients with multimorbidity. Alternative embodiments implementing monitoring and intervention include using mobile apps or voice-controlled speech interface devices to access cloud control services capable of processing automated voice recognition-response and natural language understanding-processing to perform functions and fulfill user requests.

### RELATED APPLICATIONS

This application claims the benefit of the following U.S. Provisional Patent Applications: U.S. Provisional Application 62/540,456, entitled “SYSTEM FOR THERAPY ADHERENCE INTERVENTION,” filed Aug. 2, 2017 and hereby incorporated by reference; U.S. Provisional Application 62/544,369, entitled “ASSISTIVE SYSTEM FOR SELF-MANAGEMENT OF CHEMOTHERAPY,” filed Aug. 11, 2017 and hereby incorporated by reference; U.S. Provisional Application 62/547,519, entitled “ASSISTIVE SYSTEM FOR SELF-MANAGEMENT OF ANTICOAGULANT THERAPY,” filed Aug. 18, 2017 and hereby incorporated by reference; U.S. Provisional Application 62/551,007, entitled “ASSISTIVE DIGITAL THERAPEUTIC PAIN MANAGEMENT SYSTEM,” filed Aug. 28, 2017 and hereby incorporated by reference; U.S. Provisional Application 62/558,936, entitled “ASSISTIVE PATIENT-CENTERED MEDICAL HOME SYSTEM FOR THE MANAGEMENT OF PATIENTS WITH MULTIMORBIDITY,” filed Sep. 15, 2017 and hereby incorporated by reference.

### FIELD

The present disclosure relates to the field of telemedicine and connected healthcare and health delivery devices; in particular, a patient-centered assistive system for multi-therapy adherence intervention and care management.

### BACKGROUND

Primary care, including targeting disease prevention and management, care coordination, and patient engagement, is critical to improving health and outcomes as well as controlling the costs of care. The patient-centered medical home (PCMH) is a care delivery model frequently touted as providing the structure to support this type of practice. PCMH is a concept of a team of providers caring for patients with the goals to improve the quality of care while simultaneously decreasing cost. The characteristics of a PCMH include a place of care integration, family and patient partnership and engagement, and the incorporation of primary care core attributes of personal, first contact access, comprehensive, and coordinated care.

In response to the changing patient presentations, PCMH has undergone structural and process transformations designed to facilitate care coordination and management of patients with multimorbidity, defined as the coexistence of multiple chronic conditions or diseases, increasingly common in the aging population. Multimorbidity has a significant impact on healthcare utilization and costs, affects quality of life (QoL), ability to work, employability, disability, processes of care and mortality. Multimorbidity also increases risk of polypharmacy and the complexity of treatment regimens. Despite the burden of multimorbidity, patients often receive care that is fragmented, incomplete, inefficient, and ineffective resulting in challenges for people receiving care they need from primary care. Fragmentation often occurs because programs/models and treatment strategies are typically focused on single discordant chronic conditions (e.g., diabetes, cancer, dementia, or etc.) rather than offering comprehensive approaches to simultaneously manage multiple conditions. Thus, existing approaches based on the single disease paradigm appear increasingly inappropriate for the growing number of patients with multimorbidity.

Primary patient-centered care is a cornerstone of the PCMH model. This model often defines primary care as a comprehensive, first-contact, acute, chronic, and preventive care across the life span, delivered by a team of individuals led by the patient's personal physician. The model encompasses the essential function of care coordination across multiple settings and clinicians. It encourages active engagement of patients at all levels of care delivery, ranging from shared decision-making to practice improvement. This involves a significant cultural change whereby the patient is an active, prepared, and knowledgeable participant in their care. There is a need for greater use of shared decision-making tools to assess patient preferences for different treatment options. Most medical home models incorporate the Chronic Care Model for which there is growing evidence of positive effects on chronic disease outcomes.

The PCMH model is defined by the provision of patient-centered, team-oriented, coordinated, and comprehensive care that places the patient at the center of the health care system by expanding access and improving options and alternative forms for patient-clinician communication (e.g., telephone, electronic communication, Internet “visits”). The use of telephone visits, when appropriate as a substitute for in person care, has been increasingly incorporated into the PCMH model. PCMH interventions have been suggested to lead to a reduction in multiple emergency department (ED) visits and to an increase in virtual visits for patients. Primary care providers/physicians (PCPs) play a central coordinating role in this new model. The provision of primary care can extend beyond the traditional examination room and conventional office hours, for a variety of patient populations. However, PCPs do so amidst workforce constraints and practice challenges, including adopting process improvements and electronic registries for care management, expanding care teams, offering patients, at a cost to the practice, longer in-person visits and access to electronic or telephone visits, and extending night and/or weekend business hours to enhance care access.

Unfortunately, patients with multiple chronic diseases often report poor communications with PCPs. Communication around patient needs is affected by power asymmetries between PCPs and patients because PCPs often drive the agenda. PCPs, trained primarily to elicit patients' concerns that lead to diagnosis, frequently pay less attention to the patient's daily personal experiences and challenges, particularly when patients have multiple chronic diseases. In addition, these patients also have a difficult time knowing how and when to discuss their multiple day-to-day challenges and when to discuss them, recognize or consider trade-offs between multiple competing challenges. The context in which encounters between patients and their PCPs occur can also lead to suboptimal approaches to care. These encounters usually take the form of 15-minute, multi-agenda visits, and such an approach limits the provision of optimal care and supports for self-management, as well as efforts to engage patients and PCPs in collaborative decision-making. In addition, patients with multimorbidity often see multiple healthcare providers in different settings, which may increase the risks of errors and poor care coordination. Multimorbidity also places a heavy burden on patients and caregivers for managing their care. People with multimorbidity have greater self-care needs, and older patients with complex conditions are more likely to rely on informal/family caregivers. The burden for patients and caregivers may take various forms such as managing multiple appointments with multiple healthcare professionals in multiple settings, following multiple and complex treatment regimens, as well as the stress generated by the increased burden.

Team-based approaches to healthcare delivery are tailored to address the needs of individual patients via enhanced communication. Team-based care offers many advantages including expanded access to care and more effective and efficient delivery of additional services that are essential to providing high-quality care, such as patient education, behavioral health, self-management support, and care coordination. However, the current approach to PCMH places unique burdens on both patients and healthcare team/providers. As primary care practices increasingly adopt a team-based model of care, a significant challenge is to provide an optimal IT infrastructure and processes required to facilitate and sustain effective communication among provider team members, patients, caregivers, and family members. Effective communication is essential in ensuring that care is continuous and patient-centered, as well as coordinated and coherent.

In addition to the challenges associated with PCMH in caring for patients with multimorbidity, similar challenges exist for therapy adherence intervention in specific diseases (e.g., diabetes), patient-managed therapies such as certain elements of chemotherapy, anticoagulant therapy, and pain management. For example, in the context of diabetes, patient education and promoting self-care have long been recognized as essential components in the management of diabetes and improving outcomes. Adherence to a complex medication regimen is a key component of self-care, improving blood sugar, blood pressure, and cholesterol, whereby non-adherence is associated with suboptimal glycemic control. Medication adherence commonly refers to the extent that a patient takes medications, conformance to timing, dosage, and frequency, during a prescribed length of time. Good adherence is associated with a reduction in the risk of complications, mortality, and economic burden. However, a substantial proportion of diabetics, specifically type 2s, do not take medication as prescribed; only 67-85% of oral medication doses taken, and approximately 60% of insulin doses. Non-adherence represents a complex interaction of issues and an array of social, personal, clinical, educational, and other factors contributing to sub-optimal behavior. Likewise, the therapeutic outcome of cancer treatment for patients taking oral chemotherapy agents (OAs) depends heavily on self-management. It is well documented that patients with cancer have suboptimal adherence to OAs leading to less effective treatment. Four factors have been identified as the most important barriers to oral chemotherapy adherence: complexity of medication regimens, symptom burden, poor self-management of side effects (adverse events), and low clinician support. Many OA dosing regimens require taking medication multiple times a day, cycling on and off, or taking multiple medications. In addition, the majority (^{˜}75%) of patients with cancer have comorbidities, which may interfere with the ability to self-manage. Patients with poor adherence are more likely to experience worse clinical outcomes, poor care quality, morbidity, recurrence, and mortality. Poor adherence can result in drug resistance, low response rate, earlier and more frequent disease progression, and a greater risk of death. Non-compliance may also negatively affect healthcare providers' ability to determine treatment safety and efficacy. High toxicity profile of oral chemotherapy is also a concern. Medication adherence has been found to decrease with long term medication use which may be problematic for many types of cancers that require long-term treatment. It is progressively uncovered that adherence has an important impact on pharmacokinetics (PK), pharmacodynamics (PD), and PK/PD relationships of long-term administered drugs.

Therefore, the need exists for a comprehensive solution to support the delivery of the PCMH model. The ideal system should be integrated and incorporate an optimal infrastructure to support the domains of the PCMH model (i.e., access, continuity, coordination, teamwork, comprehensive care, self-management, communication, shared decision-making). Furthermore, in applications such as therapy adherence intervention, the need exists for a digital intervention system that improves medication adherence and ultimately patient wellness in the management of diseases, including diabetes and cancer. The digital intervention should be an integrated system that incorporates comprehensive and optimal methods of improving adherence behavior including: patient education; goal-setting; feedback; behavioral skills; self-rewards; social support; interactive voice recognition-response; and others. The system should be patient-centered, comprehensive, coordinated, accessible 24/7, and committed to quality and safety. Such a system should enable a voluntary, active, and collaborative effort between patients (e.g., patients with multimorbidity), health care team/providers, caregivers, and family members, in a mutually beneficial manner to improve safety, clinical outcomes, QoL, as well as to reduce the burden of morbidities, mortality, and cost.

### SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

In the broadest terms, the invention is a pervasive integrated assistive technology platform (system) incorporating one or more computing devices, microcontrollers, memory storage devices, executable codes, methods, software, automated voice recognition-response device, automated voice recognition methods, natural language understanding-processing methods, algorithms, risk stratification tools, and communication channels for patient-centered medical home (PCMH) management of patients; including patients with specific conditions or diseases (e.g., diabetes or cancer) or multiple chronic conditions or diseases (i.e., multimorbidity). The system incorporates an optimal IT infrastructure that is patient-centered, comprehensive, coordinated, accessible 24/7, and committed to quality and safety. The intervention may be implemented in the form a wearable device providing one or more features of medication adherence, voice, data, SMS reminders, alerts, location via SMS, and 911 emergency. The device may function in combination with an application software platform accessible to multiple clients (users) executable on one or more remote servers to provide patients and caregivers with support and monitoring as well as information for a healthcare team (e.g., physicians, nurses, educators, pharmacists, etc.); a PCMH wellness ecosystem. The device may function in combination with one or more remote servers, cloud control services capable of providing automated voice recognition-response, natural language understand-processing, applications for predictive algorithm processing, sending reminders, alerts, sending general and specific information for the management of patients with multimorbidity or specific diseases or conditions. One or more components of the mentioned system may be implemented through an external system that incorporates a stand-alone speech interface device in communication with a remote server, providing cloud-based control service, to perform natural language or speech-based interaction with the user. The stand-alone speech interface device listens and interacts with a user to determine a user intent based on natural language understanding of the user's speech. The speech interface device is configured to capture user utterances and provide them to the control service. The control service performs speech recognition-response and natural language understanding-processing on the utterances to determine intents expressed by the utterances. In response to an identified intent, the controlled service causes a corresponding action to be performed. An action may be performed at the controlled service or by instructing the speech interface device to perform a function. The combination of the speech interface device and one or more applications executed by the control service serves as a relational agent. The relational agent provides conversational interactions, utilizing automated voice recognition-response, natural language processing, predictive algorithms, and the like, to perform functions, interact with the user (i.e., patient), fulfill user requests, educate and inform user, monitor user compliance, manage user symptoms, determine user health status, user well-being, suggest corrective actions-behaviors, and the like.

In a preferred embodiment, the wearable device's form-factor is a hypoallergenic wrist watch, a wearable mobile phone, incorporating functional features that include, but are not limited to, medication reminder, voice, data, SMS text messaging, fall detection, step counts, location-based services, and direct 911 emergency access. In an alternative embodiment, the wearable device's form factor is an ergonomic and attachable-removable to-and-from an appendage or garment of a user as a pendant or the like. The wearable device may contain one or more microprocessor, microcontroller, micro GSM/GPRS chipset, micro SIM module, read-only memory device, memory storage device, I-O devices, buttons, display, user interface, rechargeable battery, CODEC, microphone, speaker, wireless transceiver, antenna, accelerometer, vibrating motor(output), preferably in combination, to function fully as a wearable mobile cellular phone. The said device enables communication with one or more remote servers capable of providing automated voice recognition-response, natural language understand-processing, predictive algorithm processing, reminders, alerts, general and specific information for the management of patients with multimorbidity and/or specific diseases or conditions. One or more components of the mentioned system may be implemented through an external system that incorporates a stand-alone speech interface device in communication with a remote server, providing cloud-based control service, to perform natural language or speech-based interaction with the user. The said device enables the user (i.e., patients with one or more chronic conditions or diseases) to access and interact with the said relational agent for self-management that includes, but is not limited to, personalizing treatment plans, personalizing dosing regimens, receiving medication reminders, scheduling visits, reporting symptoms/adverse events, accessing educational information, accessing social support, and communicating with caregivers and a healthcare team (i.e., clinicians, PCPs, nurses, educators, etc.).

In another preferred embodiment, the wearable device can communicate with a secured HIPAA-compliant remote server. The remote server is accessible through one or more computing devices, including but not limited to, desk-top, laptop, tablet, mobile phone, smart appliances (e.g., smart TVs), and the like. The remote server contains a wellness support application software that include a database for storing patients and user(s) information. The application software provides a collaborative working environment to enable a voluntary, active, and collaborative effort between patients, health care team/providers, caregivers, and family members, in a mutually beneficial manner to improve efficacy, achieve therapeutic targets, improve clinical outcomes, improve QoL, as non-pharmacologic prophylaxis, as well as to reduce morbidity, recurrence, and cost. The software environment allows for, but is not limited to, daily tracking of patient location, monitoring of medication adherence, storing and tracking health data (e.g., blood pressure, glucose, cholesterol, etc.), storing symptoms (e.g., pain, etc.), displaying symptom trends and severity, sending-receiving text messages, sending-receiving voice messages, sending-receiving videos, streaming instructional videos, scheduling doctor's appointments, patient education information, caregiver education information, feedback to healthcare providers, and the like. The application software can be used to store skills relating to the self-management of specific chronic conditions or diseases. The application software may contain functions for predicting patient behaviors, functions predicting, non-compliance to diagnostic monitoring, non-compliance to pharmacologic therapy, non-compliance to physical therapy, functions for predicting symptom trends, functions for suggest corrective actions, functions to perform or teach non-pharmacologic interventions. The application software may interact with an electronic health or medical record system.

In an alternative embodiment, the said secured remote server is accessible using said stand-alone speech interface device or the speech interface is incorporated into one or more smart appliances, or mobile apps, capable of communicating with the same or another remote server, providing cloud-based control service, to perform natural language or speech-based interaction with the user, acting as said relational agent. The relational agent provides conversational interactions, utilizing automated voice recognition-response, natural language learning-processing, perform various functions and the like, to: interact with the user, fulfill user requests, educate, monitor compliance, monitor persistence, provide one or more skills, ask one or more questions, store responses/answers, perform predictive algorithms with user responses, determine health status and well-being, and provide suggestions for corrective actions including instructions for non-pharmacologic interventions.

In yet another embodiment, skills are developed and accessible through the relational agent. These skills include disease specific educational topics, nutrition (e.g., glycemic index, etc.), instructions for taking medication, instructions for point-of-care testing (POCT)-monitoring, skills to improve medication adherence, skills to increase persistence, skills for managing symptoms, proprietary developed skills, skills developed by another party, patient coping skills, behavioral skills, skills for daily activities, skills for caring for patients with multimorbidity, skills for caring for patients prescribed or taking OAs, and other skills disclosed in the detailed embodiments of this invention.

In yet another embodiment, the user interacts with the relational agent via providing responses or answers to clinically validated questionnaires or instruments. The questionnaires enable the monitoring of patient behaviors, POCT compliance, medication compliance, medication adherence, medication persistence, wellness, symptoms, adverse events monitoring, and the like. The responses or answers provided to the relational agent serve as input to one or more predictive algorithms to calculate a risk stratification profile and trends. Such a profile can provide an assessment for the need of any intervention required by either the patient, healthcare team/providers, caregivers, or family members.

In summary, the pervasive integrated assistive technology platform enables a high level of interaction for patients with multimorbidity, healthcare team/providers, caregivers, and family members. The system leverages a voice-controlled empathetic relational agent for patient education, patient support, patient social contact support, support of daily activities, patient safety, symptoms management, support for caregivers, feedback for healthcare team/providers, and the like, in the management of chronic conditions and diseases to achieve target therapeutic goals, improve QoL and quality of care, as well as to reduce complications, hospitalization, ED visits, burden of morbidity, recurrence, and cost.

Still further, an object of the present disclosure is a patient-centered assistive system for therapy adherence intervention and care management, the system comprising a speech interface device operably engaged with a communications network, the speech interface device being configured to receive a voice input from a patient user, process a voice transmission in response to receiving the voice input, and communicate the voice transmission over the communications network pursuant to at least one communications protocol, the speech interface device having at least one audio output means; and, a remote server being operably engaged with the speech interface device via the communications network to receive the voice transmission, the remote server executing a control service comprising an automated speech recognition function, a natural-language processing function, and an application software, the natural-language processing function processing the voice transmission to deliver one or more inputs to the application software, the application software executing one or more routines in response to the one or more inputs, the one or more routines comprising instructions for delivering one or more self-management prompts to the patient user via the speech interface device.

Another object of the present disclosure is a patient-centered assistive system for therapy adherence intervention and care management, the system comprising a speech interface device operably engaged with a communications network, the speech interface device being configured to receive a voice input from a patient user, process a voice transmission from the voice input, and communicate the voice transmission over the communications network pursuant to at least one communications protocol, the voice transmission defining a patient interaction, the speech interface device having at least one audio output means; a remote server being operably engaged with the speech interface device via the communications network to receive and store the voice transmission, the remote server executing a control service comprising an automated speech recognition function, a natural-language processing function, and an application software, the control service processing the voice transmission to deliver one or more inputs to the application software, the application software executing one or more routines in response to the one or more inputs, the one or more routines comprising instructions for delivering one or more self-management prompts to the patient user via the speech interface device; and, a non-patient interface device being operably engaged with the remote server via the communications network to receive health status and communications associated with the patient user, the non-patient interface device being operable to configure the one or more self-management prompts and send one or more communications to the patient interface device.

Yet another object of the present disclosure is a patient-centered assistive system for therapy adherence intervention and care management, the system comprising a speech interface device operably engaged with a communications network, the speech interface device being configured to receive a voice input from a patient user, process a voice transmission from the voice input, and communicate the voice transmission over the communications network pursuant to at least one communications protocol, the voice transmission defining a patient interaction, the speech interface device having at least one audio output means; a remote server being operably engaged with the speech interface device via the communications network to receive and store the voice transmission, the remote server executing a control service, the control service comprising an automated speech recognition function, a natural-language processing function, a user management function, and an application software, the control service processing the voice transmission to deliver one or more inputs to the application software, the application software executing one or more routines in response to the one or more inputs, the one or more routines comprising instructions for delivering one or more self-management prompts to the patient user via the speech interface device; a non-patient interface device being operably engaged with the remote server via the communications network to receive health status and communications associated with the patient user, the non-patient interface device being operable to configure the one or more self-management prompts and send one or more communications to the patient interface device; and, one or more third-party application servers being communicably engaged with the remote server via an application programming interface or abstraction layer, the one or more third-party application servers being operably engaged with the remote server to enable one or more system skills.

The foregoing has outlined rather broadly the more pertinent and important features of the present invention so that the detailed description of the invention that follows may be better understood and so that the present contribution to the art can be more fully appreciated. Additional features of the invention will be described hereinafter which form the subject of the claims of the invention. It should be appreciated by those skilled in the art that the conception and the disclosed specific methods and structures may be readily utilized as a basis for modifying or designing other structures for carrying out the same purposes of the present invention. It should be realized by those skilled in the art that such equivalent structures do not depart from the spirit and scope of the invention as set forth in the appended claims.

### BRIEF DESCRIPTION OF DRAWINGS

The above and other objects, features and advantages of the present disclosure will be more apparent from the following detailed description taken in conjunction with the accompanying drawings, in which:

FIG. 1 is a system diagram of the pervasive integrated assistive technology system incorporating a portable mobile device;

FIG. 2 is a system diagram of the pervasive integrated assistive technology system incorporating a wearable mobile device;

FIG. 3 is a perspective view of a wearable device and key features;

FIG. 4 depicts an alternate wearing option and charging function of the wearable device;

FIG. 5 is a graphical user interface containing the features of an application software platform providing a PCMH wellness ecosystem for implementing the pervasive assistive technology system;

FIG. 6 depicts a graphical user interface of the application software platform accessed through a multimedia player-television using a voice-activated speech interface remote controlled device;

FIG. 7 illustrates the pervasive integrated assistive technology system incorporating a multimedia device; and,

FIG. 8 is a functional block diagram of the elements of a relational agent.

### DETAILED DESCRIPTION

Embodiments of the present invention will now be described more fully hereinafter with reference to the accompanying drawings, in which some, but not all, embodiments of the invention are shown. Indeed, the invention may be embodied in many different forms and should not be construed as limited to the embodiments set forth herein; rather, these embodiments are provided so that this disclosure will satisfy applicable legal requirements. Where possible, any terms expressed in the singular form herein are meant to also include the plural form and vice versa, unless explicitly stated otherwise. Also, as used herein, the term “a” and/or “an” shall mean “one or more,” even though the phrase “one or more” is also used herein. Furthermore, when it is said herein that something is “based on” something else, it may be based on one or more other things as well. In other words, unless expressly indicated otherwise, as used herein “based on” means “based at least in part on” or “based at least partially on.” Like numbers refer to like elements throughout.

It is an object of the present invention to establish a PCMH wellness ecosystem that enables the implementation of, preferably, but not limited to, the Chronic Care Model. The

Chronic Care Model preferably combines the following features: self-management support (i.e., empowering and preparing patients to manage their health and healthcare); decision support (i.e., promoting clinical care that is consistent with scientific evidence and patient preferences); delivery system design (i.e., organizing programs and services to assure the proactive, effective, efficient clinical care and self-management support by healthcare teams); clinical information systems (i.e., organizing patient/population data to facilitate more efficient care); and an advanced health system (i.e., creating mechanisms that promote safe, and high quality care).

This disclosure describes a pervasive integrated assistive technology platform for facilitating a high level of interaction between patients with specific conditions or diseases (e.g., diabetes or cancer) and/or multiple chronic conditions or diseases (e.g., multimorbidity), and healthcare team/providers, caregivers and family members. The system leverages a voice-controlled empathetic relational agent for patient education, patient support, patient social contact support, support of daily activities, patient safety, symptoms management, support for caregivers, feedback/communication for and between healthcare team/providers, and the like, in self-management to achieve therapeutic targets/goals, improve efficacy, clinical outcomes, QoL, quality of care, as non-pharmacologic prophylaxis, as well as reduce hospitalization/re-hospitalization, ED visits, burden of morbidity(ies), recurrence, and cost. The platform enables the optimization of PCMH to overcome barriers to medication adherence for patients and increase compliance for health and well-being. In one embodiment, the platform or system comprises a combination of at least one of the following components: communication device; computing device; communication network; remote server; cloud server; cloud application software. The cloud server and service are commonly referred to as “on-demand computing”, “software as a service (SaaS)”, “platform computing”, “network-accessible platform”, “cloud services”, “data centers”, or the like. The cloud server is preferably a secured HIPAA-compliant remote server. In an alternative embodiment, the intervention system comprises a combination of at least one; voice-controlled speech interface device; computing device; communication network; remote server; cloud server; cloud application software. These components are configured to function together to enable a user to interact with a resulting relational agent. In addition, an application software, accessible by the user and others, using one or more remote computing devices, provides an environment, an PCMH wellness ecosystem, to enable a voluntary, active, and collaborative effort between patients, health care team/providers, caregivers, and family members, in a mutually acceptable manner to improve communication, achieve therapeutic target/goals, improve safety, clinical outcomes, QoL, as well as to reduce hospitalization, ED visits, burden of morbidity (ies), mortality, recurrence, and cost.

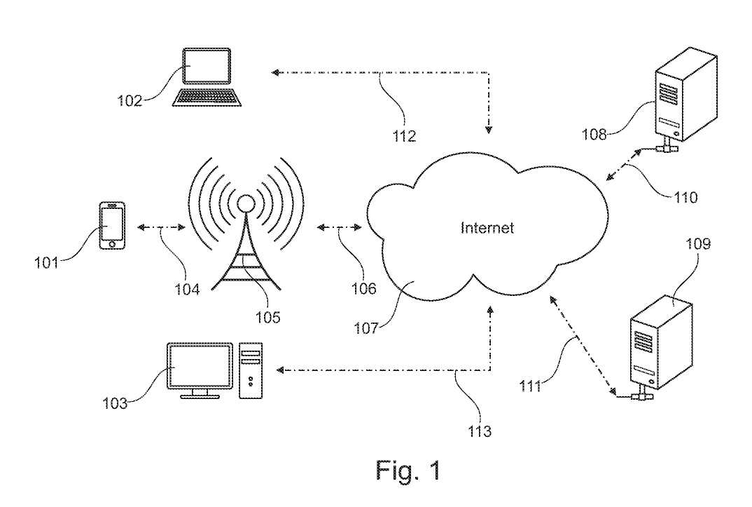

FIG. 1 illustrates the pervasive integrated assistive technology system incorporating a portable mobile device 101 for a patient with one or more chronic conditions or diseases or the like to interact with one or more remote healthcare team/provider, caregiver, or family member. One or more user can access the system using a portable computing device 102 or stationary computing device 103. Device 101 communicates with the system via communication means 104 to one or more cellular communication network 105 which can connect device 101 via communication means 106 to the Internet 107. Device 101, 102, and 103 can access one or more remote servers 108, 109 via the Internet 107 through communication means 110 and 111 depending on the server. Device 102 and 103 can access one or more servers through communication means 112 and 113. Computing devices 101, 102, and 103 are preferable examples but may be any communication device, including tablet devices, cellular telephones, personal digital assistant (PDA), a mobile Internet accessing device, or other user system including, but not limited to, pagers, televisions, gaming devices, laptop computers, desktop computers, cameras, video recorders, audio/video player, radio, GPS devices, any combination of the aforementioned, or the like. Communication means may comprise hardware, software, communication protocols, Internet protocols, methods, executable codes, instructions, known to one of ordinary skill in the art, and combined to as to establish a communication channel between two or more devices. Communication means are available from one or more manufacturers. Exemplary communication means include wired technologies (e.g., wires, universal serial bus (USB), fiber optic cable, etc.), wireless technologies (e.g., radio frequencies (RF), cellular, mobile telephone networks, satellite, Bluetooth, etc.), or other connection technologies. Embodiments of the present disclosure may incorporate any type of communication network to implement one or more communications protocols of the disclosed system, including data and/or voice networks, and may be implemented using wired infrastructure (e.g., coaxial cable, fiber optic cable, etc.), a wireless infrastructure (e.g., RF, cellular, microwave, satellite, WIFI, Bluetooth, etc.), and/or other connection technologies.

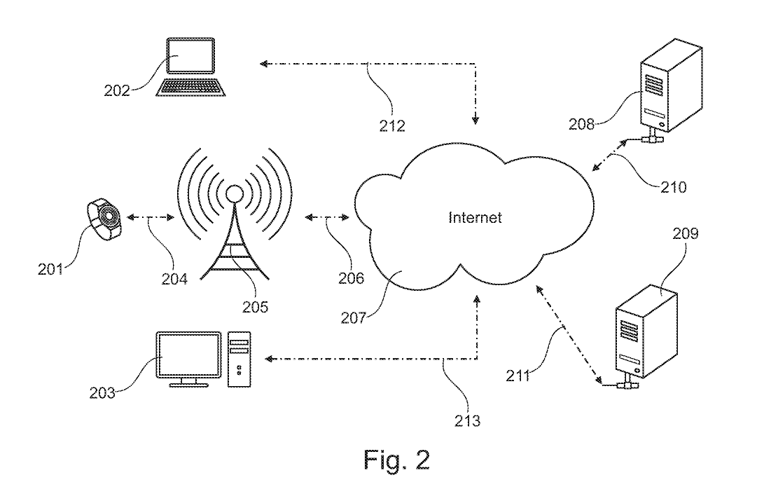

FIG. 2 illustrates the pervasive integrated assistive system incorporating a wearable device 201 for a patient with specific conditions or diseases (e.g., diabetes or cancer) and/or multimorbidity or the like to interact with one or more remote healthcare team/provider, caregiver, or family member. In a similar manner as illustrated in FIG. 1, one or more user can access the system using a portable computing device 202 or stationary computing device 203. Computing device 202 may be a laptop used by a family member or caregiver. Stationary computing device 203 may reside at the facility of a healthcare provider (i.e., physician's office). Device 201 communicates with the system via communication means 204 to one or more cellular communication network 205 which can connect device 201 via communication means 206 to the Internet 207. Device 201, 202, and 203 can access one or more remote servers 208, 209 via the Internet 207 through communication means 210 and 211 depending on the server. Device 202 and 203 can access one or more servers through communication means 212 and 213.

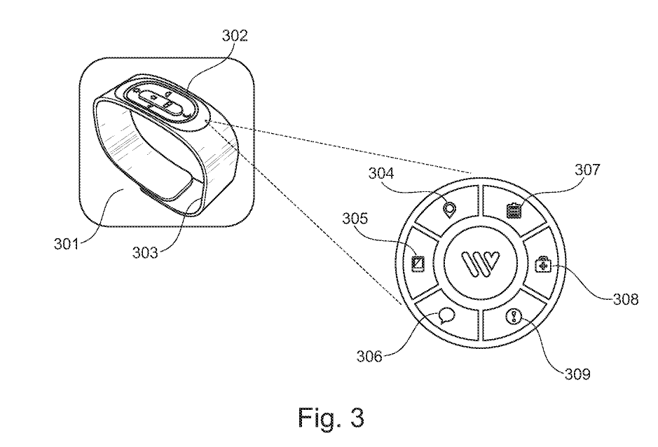

FIG. 3 is a pictorial rendering of the form-factor of a wearable device 301 as a wrist watch as a component of the pervasive integrated assistive technology system. The wearable device 301 is a fully functional mobile communication device (e.g., mobile cellular phone) that can be worn on the wrist of a user. The wearable device 301 comprises a watch-like device 302 snap-fitted onto a hypoallergenic wrist band 303. The watch-like device 302 provides a user-interface that allows a user to access features that include smart and secure location-based services 304, mobile phone module 305, voice and data 306, advanced battery system and power management 307, direct 911 access 308, and fall detection accelerometer sensor 309. The wearable may contain one or more microprocessor, microcontroller, micro GSM/GPRS chipset, micro SIM module, read-only memory device, memory storage device, I-O devices, buttons, display, user interface, rechargeable battery, CODEC, microphone, speaker, wireless transceiver, antenna, accelerometer, vibrating motor, preferably in combination, to function fully as a wearable mobile cellular phone. A patient with one or more chronic conditions/diseases or the like may use wearable device 301, depicted as device 201 of FIG. 2, to communicate with one or more healthcare team/provider, caregiver, or family member. The wearable device 301 may allow a patient to access one or more remote cloud servers to communicate with a relational agent.

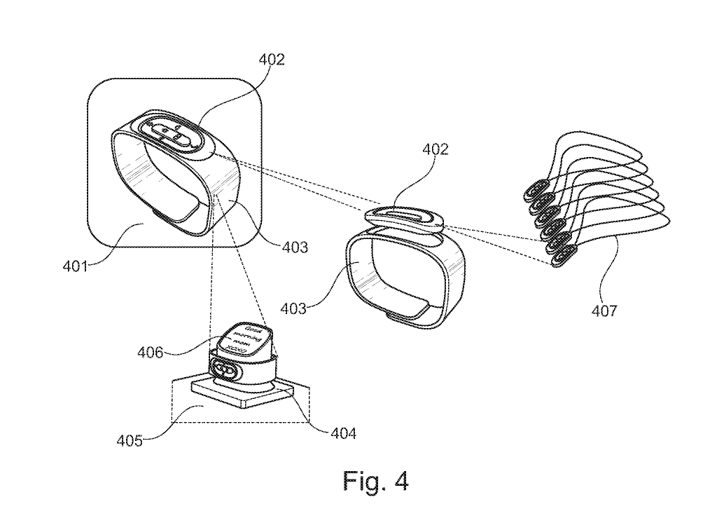

FIG. 4 illustrates details on additional features of the preferred wearable device. Wearable device 401 comprises a watch-like device 402 and wrist band 403, depicted in FIG. 3 as wearable device 301. Wearable device 401 can be stored together with a base station 404 and placed on top of platform 405. Platform 405 may be the surface of any furniture including a night stand. Base station 404 contains electronic hardware, computing devices, and software to perform various functions, for example to enable the inductive charging of the rechargeable battery of wearable device 401, among others. Base station 404 also has a user interface 406 that can display visual information or provide voice messages to a user. Information can be in the form of greetings, reminders, phone messages, and the like. Watch-like device 402 is detachable from wrist band 403 and can be attached to band 407 to be worn by a user as a necklace.

The pervasive integrated assistive technology system of the present disclosure utilizes an application software platform to enable a PCMH wellness ecosystem for patient support, patient social contact support, support of daily activities, patient safety, symptoms management, support for caregivers, feedback/communication for healthcare team/providers, and the like, in the self-management of multiple chronic conditions or diseases to improve communication, achieve therapeutic target/goals, improve safety, clinical outcomes, QoL, as well as to reduce hospitalization, ED visits, burden of morbidities, mortality, recurrence, and cost. Likewise, the application software platform may be configured to enable an ecosystem for patient support, patient social contact support, support of daily activities, patient safety, symptoms management, support for caregivers, feedback/communication for healthcare team/providers, and the like, in the self-management of specific therapies such as chemotherapy, to improve efficacy, clinical outcomes, quality of care, as well as reduce morbidity and disease reoccurrence (e.g., tumor). For therapies related to diseases such as Atrial Fibrillation (AF), Deep Vein Thrombosis (DVT), and Pulmonary Embolism (PE), the application software platform is configured to enable an ecosystem for patient support, patient social contact support, support of daily activities, patient safety, symptoms management, support for caregivers, feedback/communication for healthcare providers, and the like, in the self-management (e.g., SMW) of anticoagulant therapy to improve efficacy, achieve target international normalized ratio, reduce time to maintenance dose, increase time in therapeutic range, clinical outcomes, quality of care, prevent strokes, prevent thrombosis, as well as reduce morbidity and recurrence (e.g., stroke, AF, etc.).

The application software platform is stored in one or more servers 108, 109, 208, 209 as illustrated in FIG. 1, FIG. 2. The application software platform is accessible to users through one or more computing devices such as device 101, 102, 103, 201, 202, 203 described in this invention. Users of the application software can interact with each other via the said communication means. The software environment allows for, inter alio, daily tracking of patient location, monitoring medication adherence, monitoring persistence, storing and tracking health data (e.g., blood pressure, glucose, cholesterol, etc.), storing symptoms (e.g., bruising, bleeding, pain), displaying symptom trends and severity, sending-receiving text messages, sending-receiving voice messages, sending-receiving videos, streaming instructional videos, scheduling doctor's appointments, patient education information, caregiver education information, feedback to healthcare providers, and the like. The application software can be used to store skills relating to the self-management of specific or multiple chronic diseases. The application software may contain functions for predicting patient behaviors, functions for predicting non-compliance to therapy and POCT-monitoring, functions for predicting symptom trends, functions for suggesting corrective actions, functions to perform or teach non-pharmacologic interventions. The application software may interact with an electronic health or medical record system.

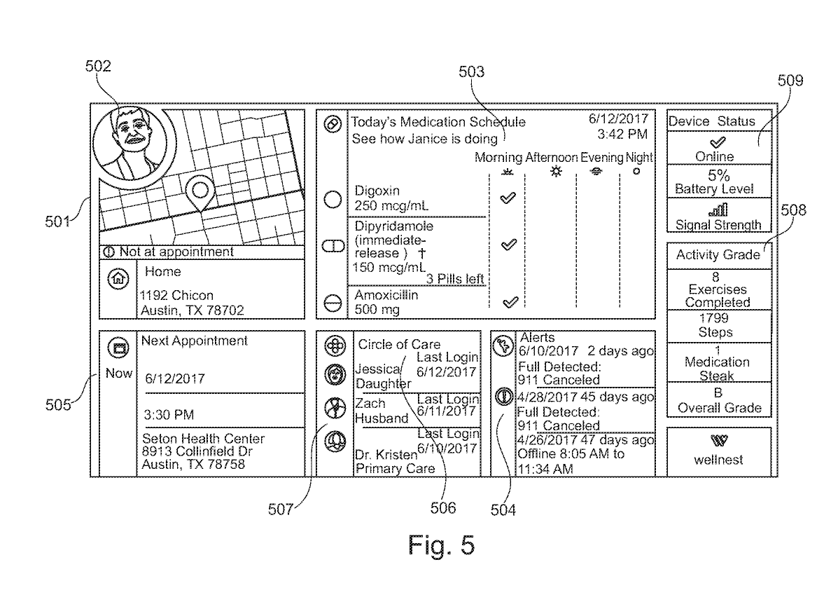

FIG. 5 is a screen-shot 501 that illustrates the type of information that users can generate using the application software platform. Screen-shot 501 provides an example of the information arranged in a specific manner and by no means limits the potential alternative or additional information that can be made available and displayed by the application software. In this example, a picture of patient 502 is presented at the upper left corner. The application may display the current location of patient 502, providing real-time location of the patient. A Medication Schedule 503 is available for review and contains a list of medications, dosage, and time when taken. This may be useful for caregivers and healthcare providers in the monitoring of patient compliance. An Alerts module 504 is also visible that documents fall detection events and 911 emergency connections for patient 502. The user can review the Next Appointment 505 information. A Circle of Care 506 has pictures of the people 507 (e.g., family members) interacting with patient 502 in this PCMH wellness ecosystem and log-in information. There's also an Activity Grade 508 that allows users to monitor for example the physical activities of patient 502 using the step count function of the wearable device. Lastly, but not least, Device Status 509 provides information on the status of said wearable device, described for example in FIG. 3, as wearable device 301.

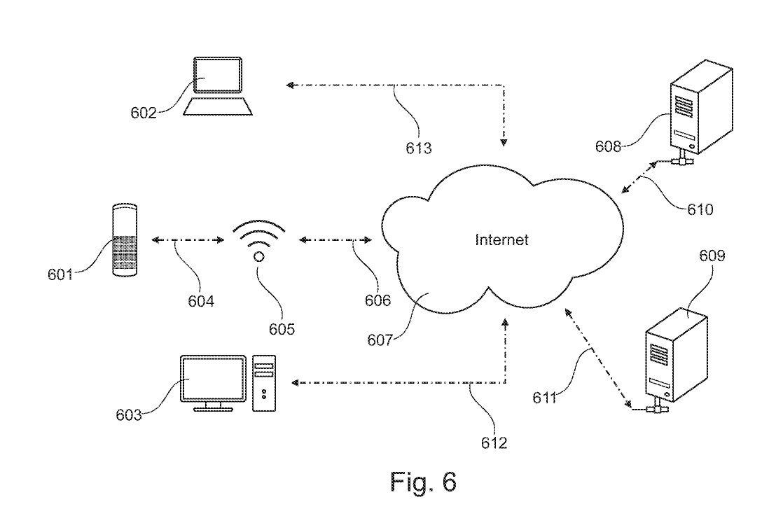

FIG. 6 illustrates the pervasive integrated assistive technology system incorporating a stand-alone voice-activated speech interface device 601 for a patient to interact with one or more remote healthcare team/provider, caregiver, or family member through a relational agent. In a similar manner as illustrated in FIG. 1, one or more user can access the system using a portable computing device 602 or stationary computing device 603. Computing device 602 may be a laptop used by a family member. Stationary computing device 603 may reside at the facility of a healthcare provider (e.g., physician's office). Device 601 communicates with the system via communication means 604 to one or more WIFI communication network 605 which can connect device 601 via communication means 606 to the Internet 607. Device 601, 602, and 603 can access one or more remote servers 608, 609 via the Internet 607 through communication means 610 and 611 depending on the server. Device 602 and 603 can access one or more servers through communication means 612 and 613. A user may request device 601 to call a family or a health care provider. Exemplary stand-alone speech interface devices with intelligent voice AI capabilities include, but are not limited to: Echo, Dot, and Show; all available from Amazon (Seattle, Wash.); Siri, Duplex, Home available from Google, Inc. (Mountain View, Calif.); Cortana available from Microsoft, Inc. (Redmond, Wash.); or the like.

In a preferred embodiment, the said stand-alone device 601 enables communication with one or more remote servers, for example server 608, capable of providing cloud-based control service, to perform natural language or speech-based interaction with the user. The stand-alone speech interface device 601 listens and interacts with a user to determine a user intent based on natural language understanding of the user's speech. The speech interface device 601 is configured to capture user utterances and provide them to the control service located on server 608. The control service performs speech recognition-response and natural language understanding-processing on the utterances to determine intents expressed by the utterances. In response to an identified intent, the controlled service causes a corresponding action to be performed. An action may be performed at the control service or by instructing the speech interface device 601 to perform a function. The combination of the speech interface device 601 and control service located on remote server 608 serve as a relational agent. The relational agent provides conversational interactions, utilizing automated voice recognition-response, natural language processing, predictive algorithms, and the like, to: perform functions, interact with the user, fulfill user requests, educate the user, monitor user compliance, monitor/track user symptoms, determine user health status, user well-being, suggest corrective user actions-behaviors, and the like. The relational agent may fulfill specific requests including calling a family member, a healthcare provider, or arrange a ride (e.g., Uber, Circulation) for the user. In an emergency, for example, a stroke event or symptoms of a heart attack, the relational agent may contact an emergency service. Ultimately the said device 601 enables the user to access and interact with the said relational agent to provide patient education, patient support, patient social contact support, support of daily activities, patient safety, symptoms management, record diary entry/contents, support for caregivers, feedback/communication for healthcare team/providers, and the like, in the self-management of multiple chronic conditions or diseases to improve communication, achieve therapeutic target/goals, improve safety, clinical outcomes, QoL, as well as to reduce hospitalization, ED visits, burden of morbidities, mortality, recurrence, and cost. The information generated from the interaction of the user and the relational agent can be captured and stored in a remote server, for example remote server 609. This information may be incorporated into the application software as described in FIG. 5, making it accessible to multi-users (e.g., healthcare team, caregiver, etc.) of the PCMH wellness ecosystem of the present disclosure.

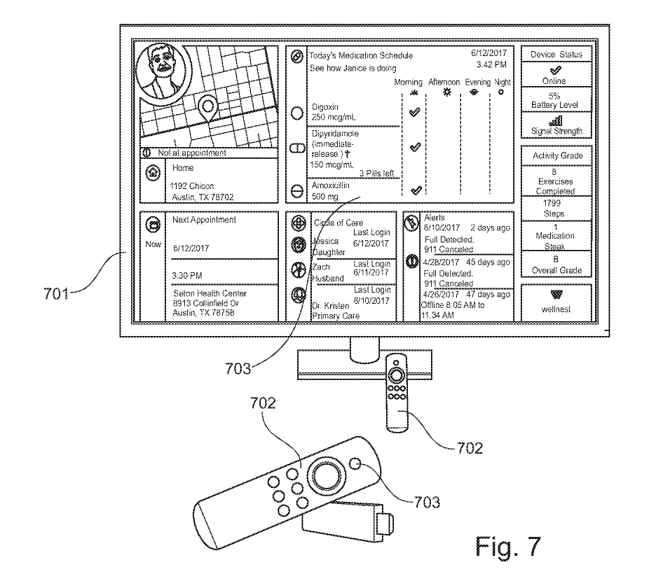

FIG. 7 illustrates the pervasive integrated assistive technology system incorporating a multimedia device 701 for a patient with at least one chronic condition/disease or the like to interact with one or more remote healthcare team/provider, caregiver, or family member through a relational agent. In a similar manner as illustrated in FIG. 6, one or more user can access the system using a remote-controlled device 702 containing a voice-controlled speech user interface 703. The multimedia device 701 is configured in a similar manner as device 601 of FIG. 6 as to enable a user to access application software platform depicted by screen-shot 704. The multimedia device 701 may be configured with hardware and software that enable streaming videos to be display. Exemplary products include FireTV, Fire HD8 Tablet, Echo Show; products available from Amazon.com (Seattle, Wash.), Nucleus (Nucleuslife.com), Triby (Invoxia.com), TCL Xcess, or the like. Streaming videos may include educational contents or materials to improve patient behavior, knowledge, attitudes, and practices to improve adherence and health outcomes. Preferable materials include contents and tools to increase patient knowledge and understanding of specific chronic condition/disease; nutritional influences on medication; the importance of regular/frequent POCT-monitoring (e.g., glucose monitoring); POCT testing techniques; therapeutic targets; physical therapy goals; symptoms, potential adverse events, and the like, and improvement of patient's planning ability and capability to incorporate the complex dosing regimens associated with the self-management of specific and/or multiple chronic conditions or diseases (e.g., multimorbidity).

In an alternative embodiment, the function of the relational agent can be accessed through a mobile app and implemented through a system illustrated in FIG.1. Such mobile app provides access to a remote, for example remote server 108 of FIG. 1, capable of providing cloud-based control service, to perform natural language or speech-based interaction with the user. The mobile app contained in mobile device 101 monitors and captures voice commands and or utterances and transmits them through the said communication means to the control service located on server 108. The control service performs speech recognition-response and natural language understanding-processing on the utterances to determine intents expressed by the utterances. In response to an identified intent, the control service causes a corresponding action to be performed. An action may be performed at the control service or by responding to the user through the mobile app. The control service located on remote server 108 serves as a relational agent. The relational agent provides a plurality of user prompts in the form of conversational interactions, utilizing automated voice recognition-response, natural language processing, predictive algorithms, and the like, to perform functions, interact with the user, fulfill user requests, educate, monitor compliance, determine health status, well-being, suggest corrective actions-behaviors, and the like. Ultimately the said device 101 enables the user to access and interact with the said relational agent for the self-management of multiple chronic conditions/diseases. The information generated from the interaction of the user and the relational agent can be captured and stored in a remote server, for example remote server 109. This information may be incorporated into the application software as described in FIG. 5, making it accessible to multi-users of the PCMH wellness ecosystem of this invention.

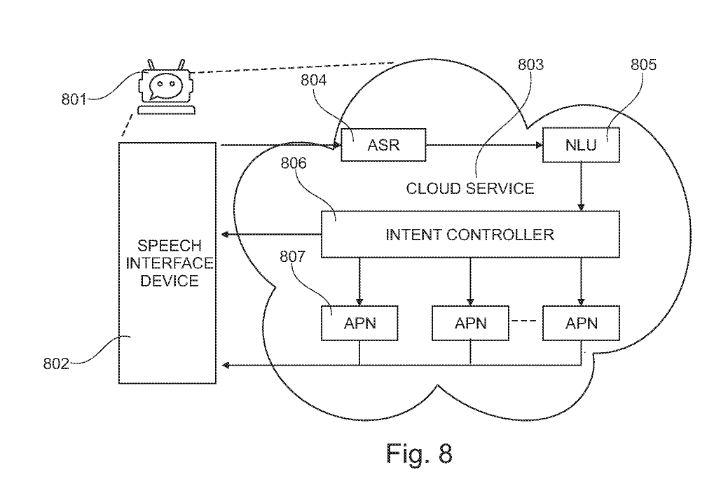

FIG. 8 illustrates a figurative relational agent 801 comprising the voice-controlled speech interface device 802 and a cloud-based control service 803. A representative cloud-based control service can be implemented through a SaaS model or the like. Model services include, but are not limited to: Amazon Web Services, Amazon Lex, Amazon Lambda, and the like, available through Amazon (Seattle, Wash.); Cloud AI, Google Cloud available through Google, Inc. (Mountain View, Calif.); Azure Al available through Microsoft, Inc. (Redmond, Wash.); or the like. Such a service provides access to one or more remote servers containing hardware and software to operate in conjunction with said voice-controlled speech interface device, app, or the like. Without being bound to a specific configuration, said control service may provide speech services implementing an automated speech recognition (ASR) function 804, a natural language understanding (NLU) function 805, an intent router/controller 806, and one or more applications 807 providing commands back to the voice-controlled speech interface device, app, or the like. The ASR function can recognize human speech in an audio signal transmitted by the voice-controlled speech interface device received from a built-in microphone. The NLU function can determine a user intent based on user speech that is recognized by the ASR components. The speech services may also include speech generation functionality that synthesizes speech audio. The control service may also provide a dialog management component configured to provide a plurality of system prompts to the user to coordinate speech dialogs or interactions with the user in conjunction with the speech services. Speech dialogs may be used to determine the user intents using speech prompts. One or more applications can serve as a command interpreter that determines functions or commands corresponding to intents expressed by user speech. In certain instances, commands may correspond to functions that are to be performed by the voice-controlled speech interface device and the command interpreter may in those cases provide device commands or instructions to the voice-controlled speech interface device for implementing such functions. The command interpreter can implement “built-in” capabilities that are used in conjunction with the voice-controlled speech interface device. The control service may be configured to use a library of installable applications including one or more software applications or skill applications of this invention. The control service may interact with other network-based services (e.g., Amazon Lambda) to obtain information, access additional database, application, or services on behalf of the user. A dialog management component is configured to coordinate dialogs or interactions with the user based on speech as recognized by the ASR component and or understood by the NLU component. The control service may also have a text-to-speech component responsive to the dialog management component to generate speech for playback on the voice-controlled speech interface device. These components may function based on models or rules, which may include acoustic models, specify grammar, lexicons, phrases, responses, and the like created through various training techniques. The dialog management component may utilize dialog models that specify logic for conducting dialogs with users. A dialog comprises an alternating sequence of natural language statements or utterances by the user and system generated speech or textual responses. The dialog models embody logic for creating responses based on received user statements to prompt the user for more detailed information of the intents or to obtain other information from the user. An application selection component or intent router identifies, selects, and/or invokes installed device applications and/or installed server applications in response to user intents identified by the NLU component. In response to a determined user intent, the intent router can identify one of the installed applications capable of servicing the user intent. The application can be called or invoked to satisfy the user intent or to conduct further dialog with the user to further refine the user intent. Each of the installed applications may have an intent specification that defines the serviceable intent. The control service uses the intent specifications to detect user utterances, expressions, or intents that correspond to the applications. An application intent specification may include NLU models for use by the natural language understanding component. In addition, one or installed applications may contain specified dialog models for that create and coordinate speech interactions with the user. The dialog models may be used by the dialog management component in conjunction with the dialog models to create and coordinate dialogs with the user and to determine user intent either before or during operation of the installed applications. The NLU component and the dialog management component may be configured to use the intent specifications of the applications either to conduct dialogs, to identify expressed intents of users, identify and use the intent specifications of installed applications, in conjunction with the NLU models and dialog modes, to determine when a user has expressed an intent that can be serviced by the application, and to conduct one or more dialogs with the user. As an example, in response to a user utterance, the control service may refer to the intent specifications of multiple applications, including both device applications and server applications, to identify a “Wellnest” intent. The service may then invoke the corresponding application. Upon invocation, the application may receive an indication of the determined intent and may conduct or coordinate further dialogs with the user to elicit further intent details. Upon determining sufficient details regarding the user intent, the application may perform its designed functionality in fulfillment of the intent. The voice-controlled speech interface device in combination with one or more functions 804, 805, 806 and applications 807 provided by the cloud service represents the relational agent 801 of the present disclosure.

In a preferred embodiment, skills are developed for the relational agent 801 of FIG. 8 and stored as accessible applications within the cloud service 803. The skills contain information that enables the relational agent to respond to intents by performing an action in response to a natural language user input, information of utterances, spoken phrases that a user can use to invoke an intent, slots or input data required to fulfill an intent, and fulfillment mechanisms for the intent. These application skills may also reside in an alternative remote service, remote database, the Internet, or the like, and yet accessible to the cloud service 803. These skills may include but are not limited to intents for general topics, weather, news, music, pollen counts, UV conditions, patient engagement skills, symptom management skills, disease specific educational topics, nutrition, instructions for taking medication, POCT (e.g., glucose monitoring, PT, aPTT, etc.), prescription instructions, medication adherence, persistence, coping skills, behavioral skills, risk stratification skills, daily activity, and the like. The skills enable the relational agent 801 to respond to intents and fulfill them through the voice-controlled speech interface device. These skills may be developed using application tools from vendors (i.e. Amazon Web Services, Alexa Skill Kits) providing cloud control services. The patient preferably interacts with relational agent 801 using skills that enable a voluntary, active, and collaborative effort between patients with multimorbidity, health care team/providers, caregivers, and family members, in a mutually beneficial manner to improve communication, achieve therapeutic target/goals, improve safety, clinical outcomes, QoL, as well as to reduce hospitalization, ED visits, burden of morbidity(ies), mortality, recurrence, and cost.

Exemplary skills accessible to a patient may be one or more non-pharmacological interventions including self-efficacy skills, self-management skills, POCT skills, medication adherence skills, symptom management skills, coping skills, and the like. It is one object of this invention to provide a relational agent with skills to be able to fulfill one or more intents invoked by a patient for example; symptoms identification (e.g., symptoms of stroke, heart attack, etc.) and management skills (e.g., invoke relational agent to call 911). It is a preferred object to utilize the spoken language interface as a natural means of interaction between the users and the system. Users can speak to the assistive technology similarly as they would normally speak to a human. It is understood, but not bound by theory, that verbal communication accompanied by the opportunity to engage in meaningful conversations can reinforce, improve, and motivate behavior for simultaneous self-management of multiple chronic conditions or diseases. The relational agent may be used to engage patients in activities aimed at stimulating social functioning to leverage social support for improving compliance, persistence, and coping. These skills may create a patient-centered environment for patient-centered care, an environment that is respectful of and responsive to the individual patient preferences, needs, values to encourage patients to value beneficial clinical decisions. Preferred skills are those, but not limited to, that examine important psychological or social constructs or utilize informative tools for assessing and improving patient symptoms, functioning, while reducing anxiety, distress, and negative behaviors. The preferred environment is a collaborative relationship between providers and patients with shared decision-making and a personal systems approach; both have the potential to improve medication adherence and patient outcomes.

The relational agent and one or more skills may be implemented in the engagement of a patient at an ambulatory setting (e.g., home, physician's office, clinic, etc.). During a session, the relational agent using one or more skills may inform the patient about a specific condition or disease. The patients may receive extensive training in POCT value self-monitoring in combination with a POCT meter. The patient may be encouraged and reminded to measure diagnostic values routinely and may record the results and the therapeutic dosages using the assistive technology platform of the present disclosure. Skills may include topics of instructions to prevent complications and the effect of diet and additional medication on the control of therapeutic targets (e.g., insulin, AC1, etc.). The relational agent may instruct the patient about indications and models of reducing or increasing therapeutic dosages to achieve target diagnostic values (e.g., blood glucose concentrations) within the target range. The relational agent may inform the patient about the possible problems that might be encountered with operations, illness, exercise, pregnancy, and traveling. The platform of this invention preferably allows the remote monitoring by a healthcare team/provider (e.g., nurse, clinician, specialist, PCP, etc.) on quality (e.g., mean, standard deviation, frequency, etc.) of POCT self-monitoring by patients and intervention as necessary.

For diseases such as Atrial Fibrillation (AF), Deep Vein Thrombosis (DVT), and Pulmonary Embolism (PE), the relational agent and one or more skills may be implemented in the engagement of a patient prescribed OACs at an ambulatory setting (e.g., home, clinic, etc.). During a session, the relational agent using one or more skills may inform the patient about anticoagulation in general. The patients may receive extensive training in INR value self-monitoring in combination with a whole blood PT/INR monitor. The patient may be encouraged and reminded to measure INR values routinely and may record the results and the anticoagulant dosages using the assistive technology platform of the invention. Skills may include topics of instructions to prevent bleeding and thromboembolic complications and the effect of diet and additional medication on anticoagulation control. The relational agent may instruct the patient about indications and models of reducing or increasing the anticoagulant dosages to achieve INR values within the target range. The relational agent may inform the patient about the possible problems that might be encountered with operations, illness, exercise, pregnancy, and traveling. Embodiments of the present disclosure preferably allow the remote monitoring by a healthcare provider (e.g., nurse, clinician) on quality (e.g., mean, standard deviation, frequency, etc.) of INR value self-monitoring by patients and intervention as necessary.

It is also an object of the present invention to provide a means to assess knowledge of specific diseases or conditions, monitor medication adherence, and assess the emotional well-being of patients with multimorbidity using a standard set of validated questionnaires and/or patient-reported outcomes (PROs) instruments. The responses-answers provided or obtained from these questionnaires and instruments enable the assessment of patient symptoms (e.g., bruising, bleeding, arrhythmia, stroke), physical functioning, psychological functioning, and overall health-related QoL. One or more questionnaires and answer-responses may be self-efficacy or confidence in the ability to perform POCT, manage disease-related symptoms or complications. This may be implemented using clinically validated questionnaires conducted by the relational agent. Upon a user intent, the relational agent can execute an algorithm or a pathway consisting of a series of questions that proceed in a state-machine manner, based upon YES or NO responses, or specific response choices provided to the user. For example, a clinically validated structured multi-item, multidimensional, questionnaire scale may be used to assess knowledge of POCT monitoring, health conditions, or symptoms, self-efficacy, and the like. The scale is preferably numerical, qualitative or quantitative, and allows for concurrent and predictive validity, with high internal consistency (i.e., high Cronbach's alpha), high sensitivity and specificity. Questions are asked by the relational agent and responses may be in the form of YES/NO answers from patients or caregivers that are recorded and processed by one or more skills. Responses may be assigned a numerical value, for example YES=1 and NO=0. A high sum of YES in this case provides a measure of non-adherence. One of ordinary skill in the art can appreciate the novelty and usefulness of using the relational agent of the present invention; a voice-controlled speech recognition and natural language processing combined with the utility of validated clinical questionnaire scales or PROs instruments. The questionnaire scales are constructed and implemented using skills developed through for example using the Alexa Skills Kit and or Amazon Lex. The combination of these modalities may be more conducive to eliciting information, providing feedback, and actively engaging patients and caregivers for the self-management of multiple chronic conditions or diseases. When a patient reports symptoms, the relational agent probes them to provide specific information about each symptom with multiple choice questions, and based on user responses, provides personalized management recommendations for that symptom.

Clinically validated scales and PROs instruments may be constructed to measure, assess, or monitor, but not limited to; physical well-being, social well-being, emotional well-being, functional well-being, pain, fatigue, nausea, sleep disturbance, distress, shortness of breath, loss of memory, loss of appetite, drowsiness, dry mouth, anxiety, sadness, emesis, numbness, bruising, disease specific-related symptoms, or the like; rated on the basis of their presence and severity. PROs instruments may also be constructed to measure, assess, or monitor medication, blood testing, POCT, medication administration, medication interactions, activity, diet, side effects, informing healthcare team/providers, procedures, lab monitoring, and QoL. It is understood that any clinically validated PROs instruments, modified or unmodified, for the management of one or more specific chronic condition or disease may be implemented using the present invention. All said questionnaires, PRO instruments, scales and the like can be constructed and implemented using the Alexa Skills Kit, cloud services from Amazon Web Services, Amazon Lex, Amazon Lambda, or the like, available through Amazon (Seattle, Wash.); Cloud AI, Google Cloud available through Google, Inc. (Mountain View, Calif.); Azure AI available through Microsoft, Inc. (Redmond, Wash.); or the like. Patient responses provide objective data about different aspects of education and practice that are taught and retained by the patient education. Thus, these instruments, for example, serve as a good quality control measure of patient education/counseling effectiveness. Frequently missed questions may indicate potential areas for improvement in patient education, including reinforcement of treatment guidelines as well as recommendation to contact healthcare providers for questions. In addition, the relational agent may assess the need for re-education or suggest areas for improvements to keep patients in compliance with therapy.

The said scales may be modifiable with variable number of items and may contain sub-scales with either yes/no answers, or response options, response options assigned to number values, Likert-response options, or Visual Analog Scale (VAS) responses. VAS responses may be displayed via mobile app in the form of text messages employing emojis, digital images, icons, and the like.

The results from one of questionnaire, scales, and PROs instruments may be obtained and or combined to monitor and provide support for patient education, social contact, daily activities, patient safety, support for caregivers, and feedback communication for healthcare providers in the self-management of multiple chronic conditions or diseases. Questionnaire, scales, and PROs instruments may be directed to either caregivers or patients of multimorbidity. Patient responses on the questionnaires are sent to the application software platform. The answers provided to the relational agent serve as input to one or more indices, predictive algorithms, statistical analyses, or the like, to calculate a risk stratification profile and trends. Such a profile can provide an assessment for the need of any intervention (i.e., corrective action) required by either the patient, healthcare team/providers, caregivers, or family members. Trends in these symptoms can be recorded and displayed in a graphical format within the application software platform showing users (e.g., patient, caregiver, healthcare providers) the severity of each symptom on each day. A care team can provide personalized management recommendations for a specific patient symptom using these results.

In summary, the pervasive integrated assistive technology system of this invention enables a high level of interaction between patients with multimorbidity, healthcare team/providers, caregivers, and family members. The system leverages a voice-activated/controlled empathetic relational agent for patient education, patient support, patient social contact support, support of daily activities, patient safety, symptoms management, support for caregivers, feedback for healthcare providers, and the like, in the concomitant management of multiple chronic conditions or diseases. For patients, the system supports patient needs including, but not limited to, POCT monitoring, medication adherence, symptoms management, coping, emotional support, social support, and educational information. For caregivers, the system supports needs including, but not limited to, providing information about specific conditions or diseases, and medication information, advice and emotional support, and health conditions, and health information resources. For healthcare team/providers, the system support needs including, but not limited to, patient behavior, profile, medication adherence, routine adherence, patient health status, and sharing of patient information across multiple healthcare settings (e.g., PCPs, specialists, pharmacists, etc.). The system establishes a PCMH wellness ecosystem that is patient-centered, comprehensive, coordinated, accessible (24/7), and enables healthcare team/providers to enhance quality improvement, ensuring that patients and families make informed decisions about their health. The system has utility in the PCMH management of patients with multimorbidity (e.g., two or more chronic conditions or diseases) including but not limited to; Alzheimer's disease and related dementia, Arthritis (osteoarthritis and rheumatoid), Asthma, Atrial fibrillation, Autism spectrum disorders, Cancer, COPD, Depression, Diabetes, Heart Failure, Hypertension, Ischemic Heart Disease, and Osteoporosis.

### EXAMPLE 1

### Patients with Multimorbidity

This example is intended to serve as a demonstration of the possible voice interactions between a relational agent and a patient with multimorbidity. The relational agent uses a control service, such as Amazon Lex available from Amazon.com (Seattle, Wash.). Access to skills requires the use of a device wake word (e.g. “Alexa”) as well as an invocation phrase (e.g. “Wellnest”) for skills specifically developed for a proprietary wearable device that embodies one or more components of the present disclosure. The following highlight one or more contemplated capabilities and uses of the invention:

| Feature | Sample Phrases |
|---|---|
| Onboarding Demo | “Alexa, open Wellnest” (conversation will continue) |
| Checking Messages | “Alexa, ask Wellnest if I have any messages” “Alexa, tell Wellnest to check my |
| | messages” |
| Appointment Schedule | “Alexa, ask Wellnest when my next appointment is.” “Alexa, ask Wellnest about my |
| | appointment schedule.” |
| Fire TV Video Content | “Alexa, ask Wellnest what is new on Fire TV about anti-hyperglycemic drugs” |
| | “Alexa, ask Wellnest if there is anything new on Fire TV about Hypertension” |
| | “Alexa, ask Wellnest if there is anything new on Fire TV about glucose testing” |
| | “Alexa, ask Wellnest if there is anything new on Fire TV about vitamin K diets” |
| General Help | “Alexa, tell Wellnest I need help to prepare for my up-coming annual doctor's visit” |
| | (conversation will continue) |
| Post-operation | “Alexa, ask Wellnest about my discharge” |
| | Alexa: “Are you having difficulties taking the oral anticoagulant as per guidance during |
| | discharge?” |
| Emergency Assistance | “Alexa, tell Wellnest to call 911 I might be having a heart attack” |
| | “Alexa, ask Wellnest to call an ambulance” |
| Contact Family | “Alexa, tell PCMH Wellnest to call Hannah” |
| Medication Reminders | “Alexa, tell Wellnest it's morning” “Alexa, tell Wellnest it's midday” “Alexa, tell |
| | Wellnest it's evening” “Alexa, tell Wellnest it's night time” |
| Medication Adherence | “Alexa, tell Wellnest I'd like to ask how I am doing with my medication” |
| Measure | Alexa: “I would like to ask you a several questions. Would you like to proceed?” |
| | Alexa: “Do you remember what you should do when realizing that you have |
| | forgotten to take your medicine?” |
| Symptoms Management | “Alexa, tell Wellnest I have a purplish swollen spot on my legs.” |
| | Alexa: “I can help you manage your symptoms. You may be experiencing |
| | bruising. I would need to ask you several questions. Would you like to proceed?” |
| | “Alexa, tell Wellnest I would like to know the symptoms of a stroke” |

### EXAMPLE 2

### Patients Prescribed or Taking OACs

This example is intended to serve as a demonstration of the possible voice interactions between a relational agent and a patient prescribed or taking OACs. The relational agent uses a control service, such as Amazon Lex available from Amazon.com (Seattle, Wash.). Access to skills requires the use of a device wake word (e.g., “Alexa”) as well as an invocation phrase (e.g., “Wellnest”) for skills specifically developed for a proprietary wearable device that embodies one or more components of the present invention called Wellnest. The following highlight one or more contemplated capabilities and uses of the invention:

| Feature | Sample Phrases |
|---|---|
| Onboarding Demo | “Alexa, open Wellnest” (conversation will continue) |
| Checking Messages | “Alexa, ask Wellnest if I have any messages” “Alexa, tell Wellnest to check my |
| | messages” |
| Appointment Schedule | “Alexa, ask Wellnest when my next appointment is.” “Alexa, ask Wellnest about my |
| | appointment schedule.” |
| Fire TV Video Content | “Alexa, ask Wellnest what is new on Fire TV about warfarin” |
| | “Alexa, ask Wellnest if there is anything new on Fire TV about atrial fibrillation” |
| | “Alexa, ask Wellnest if there is anything new on Fire TV about INR testing” |
| | “Alexa, ask Wellnest if there is anything new on Fire TV about vitamin K diets” |
| General Help | “Alexa, tell Wellnest I need help to prepare for my up-coming |
| | surgery” (conversation will continue) |
| | “Alexa, tell Wellnest I will be traveling” (conversation will continue) |
| Post-operation | “Alexa, ask Wellnest about my discharge” |
| | Alexa: “Are you having difficulties taking the oral anticoagulant as per guidance during |
| | discharge?” |
| Emergency Assistance | “Alexa, tell Wellnest to call 911 I might be having a stroke” |
| | “Alexa, ask Wellnest to call an ambulance” |
| Contact Family | “Alexa, tell Wellnest to call Hannah” |
| Medication Reminders | “Alexa, tell Wellnest it's morning” “Alexa, tell Wellnest it's |
| | midday” “Alexa, tell Wellnest it's evening” “Alexa, tell Wellnest |
| | it's night time” |
| Medication Adherence | “Alexa, tell Wellnest I'd like to ask how I am doing with my medication” |
| Measure | Alexa: “I would like to ask you a several questions. Would you like to proceed?” |
| | Alexa: “Do you remember what you should do when realizing that you have |
| | forgotten to take your medicine?” |
| Symptoms Management | “Alexa, tell Wellnest I have a purplish swollen spot on my legs.” |
| | Alexa: “I can help you manage your symptoms. You may be |
| | experiencing bruising. I would need to ask you several |
| | questions. Would you like to proceed?” |
| | “Alexa, tell Wellnest I would like to know the symptoms of a stroke” |

While certain exemplary embodiments have been described and shown in the accompanying drawings, it is to be understood that such embodiments are merely illustrative of, and not restrictive on, the broad invention, and that this invention not be limited to the specific constructions and arrangements shown and described, since various other changes, combinations, omissions, modifications and substitutions, in addition to those set forth in the above paragraphs, are possible. Those skilled in the art will appreciate that various adaptations and modifications of the just described embodiments can be configured without departing from the scope and spirit of the invention. Therefore, it is to be understood that, within the scope of the appended claims, the invention may be practiced other than as specifically described herein.

What is claimed is:

1. A patient-centered assistive system for therapy adherence intervention and care management, the system comprising:
a speech interface device operably engaged with a communications network, the speech interface device being configured to receive a voice input from a patient user, process a voice transmission in response to receiving the voice input, and communicate the voice transmission over the communications network pursuant to at least one communications protocol, the speech interface device having at least one audio output means; and,
a remote server being operably engaged with the speech interface device via the communications network to receive the voice transmission, the remote server executing a control service comprising an automated speech recognition function, a natural-language processing function, and an application software, the natural-language processing function processing the voice transmission to deliver one or more inputs to the application software, the application software executing one or more routines in response to the one or more inputs, the one or more routines comprising instructions for delivering one or more self-management prompts to the patient user via the speech interface device.

2. The system of claim 1 wherein the speech interface device requests and receives a plurality of voice inputs from the patient user in response to the one or more self-management prompts, the plurality of voice inputs defining a plurality of patient interactions.

3. The system of claim 2 wherein the remote server is further operable to store the plurality of patient interactions and perform one or more patient assessment protocols in response to the plurality of patient interactions.

4. The system of claim 1 wherein the one or more self-management prompts comprise instructions for management of two or more chronic conditions or diseases.

5. The system of claim 1 wherein the one or more self-management prompts comprise instructions for management of oral chemotherapy agent adherence.

6. The system of claim 1 wherein the one or more self-management prompts comprise instructions for management of pharmacological and non-pharmacological pain management therapies.

7. The system of claim 1 wherein the one or more self-management prompts comprise instructions for management of oral anticoagulant medication adherence.

8. The system of claim 1 wherein the one or more self-management prompts comprise instructions for management of pharmacological and non-pharmacological diabetes management therapies.

9. A patient-centered assistive system for therapy adherence intervention and care management, the system comprising:
a speech interface device operably engaged with a communications network, the speech interface device being configured to receive a voice input from a patient user, process a voice transmission from the voice input, and communicate the voice transmission over the communications network pursuant to at least one communications protocol, the voice transmission defining a patient interaction, the speech interface device having at least one audio output means;
a remote server being operably engaged with the speech interface device via the communications network to receive and store the voice transmission, the remote server executing a control service comprising an automated speech recognition function, a natural-language processing function, and an application software, the control service processing the voice transmission to deliver one or more inputs to the application software, the application software executing one or more routines in response to the one or more inputs, the one or more routines comprising instructions for delivering one or more self-management prompts to the patient user via the speech interface device; and,
a non-patient interface device being operably engaged with the remote server via the communications network to receive health status and communications associated with the patient user, the non-patient interface device being operable to configure the one or more self-management prompts and send one or more communications to the patient interface device.

10. The system of claim 9 wherein the self-management prompts comprise a plurality of patient user queries corresponding to one or more predetermined questionnaires or patient-reported outcomes instruments.

11. The system of claim 9 further comprising one or more third-party application servers being communicably engaged with the remote server via an application programming interface or abstraction layer.

12. The system of claim 9 wherein the speech interface device comprises a body-worn device.

13. The system of claim 10 wherein the one or more routines comprise instructions for executing a state-machine pathway to deliver one or more personalized management recommendations to the patient user via the speech interface device in response to a plurality of patient interactions associated with the plurality of patient user queries.

14. The system of claim 11 wherein the voice input from the patient user defines a patient user request, the control service being operable to communicate with the one or more third-party application servers to fulfill the patient user request.

15. The system of claim 11 wherein the one or more third-party application servers comprise an electronic medical records server.

16. A patient-centered assistive system for therapy adherence intervention and care management, the system comprising:
a speech interface device operably engaged with a communications network, the speech interface device being configured to receive a voice input from a patient user, process a voice transmission from the voice input, and communicate the voice transmission over the communications network pursuant to at least one communications protocol, the voice transmission defining a patient interaction, the speech interface device having at least one audio output means;
a remote server being operably engaged with the speech interface device via the communications network to receive and store the voice transmission, the remote server executing a control service, the control service comprising an automated speech recognition function, a natural-language processing function, a user management function, and an application software, the control service processing the voice transmission to deliver one or more inputs to the application software, the application software executing one or more routines in response to the one or more inputs, the one or more routines comprising instructions for delivering one or more self-management prompts to the patient user via the speech interface device;
a non-patient interface device being operably engaged with the remote server via the communications network to receive health status and communications associated with the patient user, the non-patient interface device being operable to configure the one or more self-management prompts and send one or more communications to the speech interface device; and,
one or more third-party application servers being communicably engaged with the remote server via an application programming interface or abstraction layer, the one or more third-party application servers being operably engaged with the remote server to enable one or more system skills.

17. The system of claim 16 wherein the speech interface device comprises a body-worn device.

18. The system of claim 16 wherein the one or more system skills comprise instructions for symptom identification or symptom management.

19. The system of claim 16 wherein the one or more routines of the application software further comprise instructions for concomitant management of multiple chronic conditions or diseases.

20. The system of claim 16 further comprising a multimedia device communicably engaged with the remote server via the communications network, the multimedia device being configured to display a graphical user interface comprising a plurality of patient health data and patient interaction data.
